(19) United States
(12) Patent Application Publication
(10) Pub. No.: US 2020/0245616 A1
(43) Pub. Date: Aug. 6, 2020

(54) A Disinfectant Composition with Extended Antimicrobial Effects

(71) Applicant: Siamons International Inc., Toronto (CA)

(72) Inventors: Peter Lea, Toronto (CA); Brian Bacik, Midhurst (CA); Jeff Hastie, Toronto (CA)

(21) Appl. No.: 16/494,135

(22) PCT Filed: Mar. 29, 2018
(86) PCT No.: PCT/CA2018/000065

§ 371 Date: Sep. 13, 2019

(30) Foreign Application Priority Data
Mar. 30, 2017 (CA) 2,962,787

(51) Int. Cl. A01N 33/12; A01N 59/26

(52) U.S. Cl. CPC A01N 33/12; A01N 59/26

(57) ABSTRACT

An aqueous disinfectant composition, comprising trisodium phosphate, sodium carbonate, sodium bicarbonate and a quaternary ammonium halide such as cetyltrimethylammonium bromide is provided. The aqueous disinfectant composition may comprise from about 0.25% to about 3.0% by weight of trisodium phosphate, from about 0.25% to about 3.0% by weight of sodium carbonate, from about 0.25% to about 3.0% by weight of sodium bicarbonate, and from about 0.001% to about 0.1% by weight of a quaternary ammonium halide. The invention also includes the use of a composition, comprising trisodium phosphate, sodium carbonate, sodium bicarbonate and a quaternary ammonium halide in the preparation of an aqueous disinfectant formulation.

### FIELD

The present invention relates to a disinfectant composition having both disinfecting efficacy within ten minutes and an extended long term antimicrobial efficacy thereafter, and uses thereof.

### BACKGROUND

The formulation of disinfecting solutions that have the ability to disinfect a wide variety of surfaces and materials is of significant importance. To this effect, much research in the fields of disinfecting agents has been performed and has resulted in several complex formulations. Many of these cleaning and disinfectant solutions have components that can be harmful to a user if ingested or brought into contact with a user's skin, thus requiring personal protective equipment to prevent such contact.

It would be advantageous to have a disinfectant solution capable of disinfecting a wide variety of surfaces and materials while encompassing only a few readily available components. In addition, it would be advantageous for the disinfecting solution to be non-toxic and not be a cause of harm upon human contact. Disinfectant formulations that make use of quaternary ammonium halides (QAH's) as active ingredients are known. QAH's are formulated in these disinfectant formulations at concentrations above 0.1% of the total formulation in order to be effective. It would be advantageous for a formula containing QAH's to have the lowest concentration of QAH's possible in order to remain effective, not only for cost but also to uphold a high safety profile for the user. It would be advantageous for the disinfectant solution to eliminate malodors without the need for the addition of scenting agents. It would also be advantageous to provide a disinfecting composition that has both a short-term disinfecting effect within ten minutes of application of the composition, and an extended long term antimicrobial effect on surfaces to which the composition is applied. It would be advantageous for the disinfectant solution to contain within the composition ingredients that facilitate cleaning of surfaces.

### SUMMARY

An aqueous disinfectant composition is provided comprising a quaternary ammonium halide and soluble polymer forming components wherein the composition has both a disinfecting effect within ten minutes of application of the composition to a surface and a long term antimicrobial effect imparted to the surface.

An aqueous disinfectant composition is provided comprising a quaternary ammonium halide having a concentration of about 0.001% to about 0.1% and soluble polymer forming components. Preferably, the concentration of the quaternary ammonium halide is from 0.01% to about 0.1% by weight of the composition.

According to another aspect, there is provided an aqueous disinfectant composition, comprising trisodium phosphate, sodium carbonate, sodium bicarbonate and a quaternary ammonium halide.

According to one aspect, the quaternary ammonium halide is cetyltrimethylammonium bromide (“CTAB”).

According to one aspect, the quaternary ammonium halide is cetyltrimethylammonium bromide.

According to an aspect, there is provided an aqueous disinfectant composition, comprising from about 0.25% to about 3.0% by weight of trisodium phosphate, from about 0.25% to about 3.0% by weight of sodium carbonate, from about 0.1% to about 3.0% by weight of sodium bicarbonate, in combination with a quaternary ammonium halide.

According to another aspect, there is provided an aqueous disinfectant composition, comprising from about 0.25% to about 3.0% by weight of trisodium phosphate, from about 0.25% to about 3.0% by weight of sodium carbonate, from about 0.1% to about 3.0% by weight of sodium bicarbonate, and from about 0.01% to about 0.1% by weight of a quaternary ammonium halide.

According to another aspect, there is provided an aqueous disinfectant composition, comprising from about 0.25% to about 3.0% by weight of trisodium phosphate, from about 0.25% to about 3.0% by weight of sodium carbonate, from about 0.1% to about 3.0% by weight of sodium bicarbonate, and from about 0.01% to about 0.1% by weight of a quaternary ammonium halide.

According to another aspect, there is provided an aqueous disinfectant composition, comprising about 0.237% by weight of sodium bicarbonate, about 0.948% by weight of sodium carbonate, about 1.185% by weight of trisodium phosphate and about 0.01% to about 0.1% by weight of a quaternary ammonium halide.

According to another aspect, there is provided the use of a composition, comprising a quaternary ammonium halide at a concentration of about 0.01% to about 0.1% by weight of the composition and soluble polymer forming components for the preparation of an aqueous disinfectant formulation.

According to another aspect, there is provided use of a composition, comprising trisodium phosphate, sodium carbonate, sodium bicarbonate and a quaternary ammonium halide for the preparation of an aqueous disinfectant formulation.

According to another aspect, there is provided the use of a composition, comprising about 0.237% by weight of sodium bicarbonate, about 0.948% by weight of sodium carbonate, about 1.185% by weight of trisodium phosphate and a quaternary ammonium halide for the preparation of an aqueous disinfectant formulation.

According to yet another aspect, there is provided the use of a composition, comprising about 0.237% by weight of sodium bicarbonate, about 0.948% by weight of sodium carbonate, about 1.185% by weight of trisodium phosphate and about 0.01% to about 0.1% by weight of a quaternary ammonium halide for the preparation of an aqueous disinfectant formulation.

According to another aspect, there is provided the use of a composition, comprising from about 0.25% to about 3.0% trisodium phosphate, from about 0.25% to about 3.0% sodium carbonate, 0.1% to about 3.0% sodium bicarbonate and about 0.5% by weight of a quaternary ammonium halide for disinfecting a surface contaminated by Aspergillus niger.

According to yet another aspect, there is provided a composition comprising from about 0.25% to about 3.0% trisodium phosphate, from about 0.25% to about 3.0% sodium carbonate, 0.1% to about 3.0% sodium bicarbonate and about 0.5% by weight of a quaternary ammonium halide for disinfecting a surface contaminated by Aspergillus niger.

### DETAILED DESCRIPTION

According to the present disclosure there is provided an aqueous disinfectant composition comprising a quaternary ammonium halide and soluble polymer forming components. The composition surprisingly has both a disinfecting effect within ten minutes of application of the composition to a surface as well as a long term antimicrobial effect imparted to the surface.

The disinfecting composition of the present disclosure preferably comprises sodium bicarbonate, sodium carbonate, and trisodium phosphate in water to form a tri-salt polymer. The composition preferably comprises from about 0.25% to about 3.0% by weight of the composition of trisodium phosphate, from about 0.25% to about 3.0% by weight of the composition of sodium carbonate, and from about 0.25% to about 3.0% by weight of the composition of sodium bicarbonate. Most preferably, the composition comprises 0.237% by weight of sodium bicarbonate, 0.948% by weight of sodium carbonate and 1.185% by weight of trisodium phosphate. The composition also comprises a quaternary ammonium halide. Preferably the quaternary ammonium halide is cetyltrimethylammonium bromide, trimethylstearylammonium chloride, alkyldimethylbenzylammonium chloride, benzethonium chloride or benzyl-dimethylhexadecylammonium chloride. Most preferably, the quaternary ammonium halide is cetyltrimethylammonium bromide.

The composition may comprise from at least about 0.001% by weight to about 0.1% of a quaternary ammonium halide. Preferably, the composition comprises at least about 0.01% to 0.1% by weight of a quaternary ammonium halide. Most preferably, the composition comprises from about 0.05% by weight to about 0.1% by weight of a quaternary ammonium halide. The balance of the composition is water.

The formula disclosed in U.S. Pat. No. 6,184,198, (“Cleaning Solution”) which describes a combination of salts in aqueous solution, and is referred to as “CMC” for the purposes of the present disclosure, comprises 0.237% NaHCO₃, 0.95% Na₂CO₃, 1.19% Na₃PO₄, 97.623% H₂O. CMC is a highly effective fungicide and fungistat. The CMC mechanism for the destruction of mold is the formation of a tri-salt polymer which encapsulates microorganisms. The encapsulating polymer contracts as the formula dries around the microorganism, eventually causing cell membrane rupture of the organisms on the treated surface (Lea, P. Ding, S. F. Lemez, S. B. Scanning, 25, 277-284, 203). By this mechanism the applied CMC may require prolonged periods of time (greater than 10 minutes) to kill the organism that has contaminated the surface.

It has surprisingly been discovered that the effectiveness of this anti-microbial solution comprising sodium carbonate, sodium bicarbonate, and trisodium phosphate in water is increased by addition of a quaternary ammonium halide such as cetyltrimethylammonium bromide. It is similarly surprising that only very low concentrations of cetyltrimethylammonium bromide or other quaternary ammonium halides of at least about 0.001% and preferably in the range of 0.01% to 0.1% by weight of the composition are required to produce an increased level of effectiveness in killing select microorganisms in combination with CMC. The effectiveness is observed as a reduction in contact time necessary to kill select microorganisms on a surface to 10 minutes or less after the composition is applied. The composition also imparts an extended long term antimicrobial effect to the surface on which it was applied in that further growth of microorganisms on the surface is prevented.

Without being bound by theory, it is believed that as the disinfectant composition of the present disclosure dries on a surface, it adheres to both porous and non-porous surfaces, forming a thin antimicrobial coating that adheres to the treated surface after application of the formula. The coating dries and remains on the surface to prevent future growth of fungi and other micro-organisms.

The disinfectant composition can be applied by various means. For example, the application method can be by fogging the solution through an approved ultra-low volume (ULV) cold fogger, through a spray nozzle, or by dampening a fabric or paper towel, either using a spray or pouring the disinfectant onto the towel, and then wiping the disinfectant onto the surface.

The disinfectant composition can be used on many surfaces including hard, non-porous surfaces and hard, semi-porous surfaces. The fungistatic and antimicrobial effect is imparted on hard, non-porous surfaces, hard semi-porous surfaces, and soft surfaces such as fabrics.

It has been found that inclusion of low levels of quaternary ammonium halides preferably in the range of 0.01% to 0.1% by weight, in particular cetyltrimethylammonium bromide, trimethylstearylammonium chloride, alkyldimethylbenzylammonium chloride, benzethonium chloride or benzyl-dimethylhexadecylammonium chloride in combination with a composition comprising sodium bicarbonate, sodium carbonate, and trisodium phosphate contributed a noticeable disinfecting effect. As set out below, experiments have been conducted on gram-positive (Staphylococcus aureus ATCC 6538 (SA 6538)) and gram negative (Pseudomonas aeruginosa ATCC 15442 (PA 15442), Salmonella choleraesuis ATCC 10708 (SC 10708)) bacteria, as well as fungal strains Trichophyton mentagrophytes ATCC 9533 (TM 9533), Penicillium variable ATCC 32333 (PV 32333), Aspergillus niger ATCC 6275 (AN 6275), and Stachybotrys chartarum ATCC 201867 (SC 201867)). ATCC refers to the American Type Culture Collection.

For the purposes of the present disclosure, the disinfectant composition comprising the soluble polymer forming compounds (CMC) and quaternary ammonium halides is referred to as CMC-Plus.

### Example 1: Effect of CMC-Plus on Representative Gram Positive and Gram Negative Bacteria and Representative Fungal Strain

In the present example, CMC-Plus is a composition comprising about 0.237% by weight of sodium bicarbonate, about 0.948% by weight of sodium carbonate, about 1.185% by weight of trisodium phosphate and concentrations of CTAB as defined in Table 2. The balance of the composition is water.

TABLE 1
Preparation of CMC-Plus (0.1% cetyltrimethylammonium
bromide (“CTAB”) Formula) used as ‘Control’

| Ingredients | |
|---|---|
| Type II water (millilitres) | 483.14 |
| NaHCO₃ (grams) | 1.19 |
| Na₂CO₃ (grams) | 4.74 |
| Na₃PO₄ (grams) | 5.93 |
| CTAB (grams) | 0.5 |
| pH | 11.47 |
| Appearance | Clear |

### Materials

Type II Water: equivalent to Reverse Osmosis (RO) Water.

Sodium Bicarbonate (NaHCO₃): USP #1 powder, Food Grade, CAS#144-55-8, FMC Corporation, Philadelphia, Pa., 18103, USA.

Sodium Carbonate (Na₂CO₃): Soda ash dense powder, Food Grade, CAS#497-19-8, General Chemical Industrial Products, Inc. Parsippany, N.J., 07054, USA. Trisodium Phosphate (Na₃PO₄): Anhydrous trisodium phosphate powder, Food Grade, CAS#7601-54-9, ICL performance Products LP, St. Louis, Mo. 63141, USA. CTAB: Cetyltrimethylammonium Bromide, Sigma-Aldrich, Cat #855820, Batch #06901CD.

All experiments were conducted according to the protocol outlined in AOAC Official Method 961.02 (Germicidal Spray Method), which is a carrier-based method used to evaluate disinfection efficacy of aerosol/pump-based spray products and liquid products for registration with regulatory agencies such as the U.S. EPA and Health Canada.

### Summary of the Test

In this method, a series of glass slides (“carriers”) were inoculated with a representative test organism and the carriers were dried under ambient conditions. The dried organisms on the surface of the carrier were then sequentially treated with the composition of the present disclosure in the form of a spray product and were exposed for a pre-determined exposure time. After exposure, the carriers were sequentially transferred to a liquid subculture medium specifically selected to neutralize the composition of the present disclosure and to recover any surviving test organism. The carriers were incubated and visually examined for the presence or absence of growth. Based on the desired disinfection claim and the requirements of the regulatory agency, the disinfection product must demonstrate kill on a pre-determined number of carriers inoculated with required test organisms. Required organisms for a disinfection claim include but are not limited to Staphylococcus aureus, Salmonella choleraesuis and Pseudomonas aeruginosa.

### Procedures and Results

### Experimental Procedure: Disinfection of Bacteria Cultures

Method Adapted to the Preparation of Bacteria Cultures from −80° C. Freshly Recovered Stock. Testing method described for testing of Staphylococcus aureus ATCC 6538 (SA 6538) and is applicable for testing of Salmonella choleraesuis ATCC 10708 (SC 10708) and Pseudomonas aeruginosa ATCC 15442 (PA 15442).
Defrosted a single cryovial at room temperature and briefly vortexed to mix. Added 10 μL of the thawed stock to a tube containing 10 mL synthetic broth and then vortexed to mix. Discarded the rest of cryovial stock.
Incubated the tube at 36 ±1° C. for 24 hours (h). Briefly vortexed the 24 h culture prior to transfer. For this final subculture step, inoculated two 20×150 mm tubes containing 10 mL synthetic broth with 10 mL per tube of the 24 h synthetic broth culture.
Incubated 48 h at 36±1° C. Using a Vortex-style mixer, mixed synthetic broth test cultures 3-4 seconds and let stand 10 minutes at room temperature before continuing. Removed the upper portion of each culture, leaving behind any debris or clumps, and transferred to a sterile tube. Titrated the culture by plating on synthetic agar plates, and diluted to 5×10⁸ Colony Forming Units (CFU) per mL.

### Carrier Inoculation for Disinfectant Testing

Inside a sterile hood, transferred 10μL of 5×10⁸ CFU/ml SA6538 onto a sterile test carrier (25×25 mm, VWR#89239-692, 5×10⁶ CFU/carrier) in the Petri dish onto 64 carriers (60 carriers for testing disinfectant formula version, 4 control slides). High volume screening studies were conducted in some cases where only 4 organism-inoculated carriers were treated at specified concentrations of CTAB. Vortex-mixed the inoculum periodically during inoculation of the carriers.
Immediately spread the inoculum uniformly over the majority of the carrier surface using a sterile loop. Did not impact the edges of the carriers. Covered dish immediately and repeated operation until 64 carriers had been prepared for the test. Once all of the carriers had been inoculated, placed in incubator at 37° C. for 40 minutes until dry.

### Test Formulae

0.85% NaCl, negative control, 2 carriers
CMC—0.1% CTAB, positive control, 2 carriers
CMC—0.01% CTAB, 60 carriers each

### Test Procedures and Results

After the test organisms were dried on the surface of the carrier, each carrier was sprayed with the respective disinfection formulae 10 times in 10 seconds at a distance of 30 cm.
Treatment interval was timed. Ten (10) minutes after each carrier had been sprayed by the disinfectant, the excess liquid was removed and the carriers were transferred in a sequentially timed fashion into the culture tubes containing 20 ml neutralizer (Letheen Broth with 0.07% Lecithin/0.5% Tween 80/0.1% Sodium Thiosulfate) to neutralize the disinfectant. Letheen Broth is a liquid medium recommended for use in qualitative procedures for testing formulas featuring quaternary ammonium compounds for antimicrobial activity.
After the carriers were deposited in the respective culture tubes, the tubes were recapped and the cultures were thoroughly resuspended.
Incubated all neutralization tubes at 36 ±1° C. for 48 hours.
Examined the results at hour 24 and hour 48. The percentage of the treated carriers showing growth after 48 hours was recorded. The results are listed in Table 2.

### Experimental Procedure (Trichophyton mentagrophytes ATCC 9533):

### Method Adapted to the Preparation of Fungal Cultures from −80° C. Freshly Recovered Stock.

Testing method described for testing Trichophyton mentagrophytes ATCC 9533 and is applicable to testing of Penicillium variable ATCC 32333, Aspergillus niger ATCC 6275, and Stachybotrys chartarum ATCC 201867.
The conidiospores were removed from the surfaces of 4 Sabouraud Dextrose Agar plates containing mature culture of TM9533 using sterile 0.85% saline solution containing 0.05% isooctylphenoxypolyethoxyethanol. The conidiospore suspension was macerated in a tissue grinder. The suspension was filtered through sterile cotton to remove the hyphae and hyphal fragments. For the disinfectant test, the suspension of conidia was adjusted to yield approximately 5.0×10⁶ conidia/ml, by dilution with saline solution (0.85% sodium chloride) using a haemocytometer and confirmed by standard plate count techniques.

### Carrier Inoculation for Disinfectant Testing

Inside a sterile hood, transferred 10 μL of 5×10⁶ conidia/ml TM9533 onto a sterile test carrier (25×25 mm, VWR#89239-692, 5×10⁴ conidia/carrier) in the Petri dish, respectively on 64 carriers. Vortex-mixed the inoculum periodically during inoculation of the carriers.
Immediately spread the inoculum uniformly over the majority of the carrier surface using a sterile loop. Did not impact the edges of the carriers. Covered dish immediately and repeated operation until 64 carriers (60 carriers for testing disinfection testing, and 4 control carriers) had been prepared for the test (60 carriers for testing disinfectant formula version, 4 control slides). High volume screening studies were conducted in some cases where only 4 organism-inoculated carriers were treated at specified concentrations of CTAB. Once all of the carriers had been inoculated, the carriers were placed in an incubator at 37° C. for 40 minutes until completely dry.

### Test Formulae

0.85% NaC1, negative control, 2 carriers
CMC—0.1% CTAB, positive control, 2 carriers
CMC—0.01% CTAB, 60 carriers

### Test Procedures and Results

After drying, each carrier was sprayed with the respective disinfection formulae 10 times in 10 seconds at a distance of 30 cm.
Timed the treatment interval. Ten (10) minutes after each carrier had been exposed to the disinfectant, the excess liquid was removed and the carriers were transferred in a sequentially timed fashion into the culture tubes containing 20 ml neutralization broth (Letheen Broth with 0.07% Lecithin /0.5% Tween 80) to neutralize the disinfectant.
After the carriers were deposited in the respective culture tubes, the tubes were recapped and the cultures were thoroughly resuspended.
Incubated all neutralization tubes at 28 ±1° C. for 14 days.
After 14 days, the cultures within the tubes were examined for growth. The percentage of the treated carriers showing growth was recorded. The results are listed in Table 2.

### Summary of Results

TABLE 2
Summary of Effect of CMC-Plus (CTAB concentrations Indicated) on
Representative Gram Positive and Gram-Negative Bacteria and Representative Fungal
Strains

| | | Staphylococcus | Pseudomonas | Salmonella | Trichophyton | Aspergillus | Penicillium | Stachybotrys |
|---|---|---|---|---|---|---|---|---|
| | | aureus | aeruginosa | choleraesuis | mentagrophytes | niger | variable | chartarum |
| Group | CTAB | Growth Rate^{a} | Growth Rate | Growth Rate | Growth Rate | Growth Rate | Growth Rate | Growth Rate |
| CMC ^{b} | 0.0010 | 100% | 0% | —^{c} | 0% | 100% | 75% | 25% |
| | 0.0025 | 100% | — | — | — | — | — | — |
| | 0.0050 | 80% | — | — | — | — | — | — |
| | 0.0075 | 40% | — | — | — | — | — | — |
| | 0.0100 | 0% | 0% | 0% | 0% | 0% | 0% | 0% |
| | 0.0250 | — | — | — | — | 0% | 0% | 0% |
| | 0.0500 | 0% | 0% | — | 0% | — | — | — |
| | 0.1000 | — | — | — | — | 0% | 0% | — |
| Control | 0 | 100% | 100% | 100% | 100% | 100% | 100% | 100% |
| (0.85% | | | | | | | | |
| NaCl) | | | | | | | | |

^{a}Growth rate refers to the number of carriers in the experiment showing surviving cultures.
^{b} CMC refers to a solution of 0.237% NaHCO₃, 0.95% Na₂CO₃, and 1.19% Na₃PO₄.
^{c}dashes (—) denotes that the experiment was not performed and information is not available.

TABLE 3
Summary of Effect of Various CTAB Concentrations in Water on Representative
Gram Positive and Gram-Negative Bacteria and Representative Fungal Strain

| | CTAB | Staphylococcus | Pseudomonas | Salmonella | Trichophyton | Aspergillus | Penicillium | Stachybotrys |
|---|---|---|---|---|---|---|---|---|
| | Conc. | aureus | aeruginosa | choleraesuis | mentagrophytes | niger | variable | chartarum |
| Group | (%) | Growth Rate^{a} | Growth Rate | Growth Rate | Growth Rate | Growth Rate | Growth Rate | Growth Rate |
| Water | 0.0010 | 100% | 100% | — | 0% | 100% | 100% | — |
| (Control) | 0.0100 | 100% | 100% | 100% | 0% | 100% | 0% | — |
| | 0.0500 | 40% | 100% | — | 0% | 100% | 0% | — |
| | 0.1000 | 40% | — | 0% | — | 100% | 0% | — |

^{a}Growth rate refers to the number of carriers in the experiment showing surviving cultures.
^{b} dashes (—) denotes that the experiment was not performed and information is not available.

The results obtained demonstrate that a concentration of 0.01% CTAB combined with CMC achieves a broad-spectrum disinfectant level of effectiveness, whereas CTAB on its own in water shows little efficacy against the same organisms until the concentration approaches 0.05% (Staphylococcus aureus). With respect to Pseudomonas, even this concentration was ineffective. The disinfecting formulation of the present disclosure prevents future fungal and bacterial growth in environments susceptible to contamination. A treatment of carriers inoculated with micro-organisms with CTAB alone (in the absence of CMC) confirms that the aqueous CTAB solutions have a much higher level of effectiveness in the presence of CMC.

It was expected that if the CTAB was replaced by QAH's of similar structure that the disinfection of selected bacteria and fungal organisms would be similarly achieved. Table 4 summarizes these results. In the case of bacteria testing, the experimental procedure was as outlined in Example 1 for disinfection of bacterial cultures. In the case of fungal organisms, the experimental procedure was as outlined in Example 1 for the preparation of fungal cultures.

TABLE 4
Summary of Disinfection Results Against Select Bacteria and Fungal Organisms.

| | | | Staphylococcus | Pseudomonas | Trichophyton | Aspergillus | Penicillium |
|---|---|---|---|---|---|---|---|
| | | Cone. | aureus | aeruginosa | mentagrophytes | niger | variable |
| | QAH | (%) | Growth Rate | Growth Rate | Growth Rate | Growth Rate | Growth Rate |
| CMC | — | — | 100% | 0% | 100% | 100% | 100% |
| Only | | | | | | | |
| CMC + | TMSAC | 0.010 | 0% | 0% | 0% | | 0% |
| QAH | | 0.100 | 0% | 0% | 0% | | 0% |
| | | 1.000 | 0% | 0% | 0% | | 0% |
| | BTEAC | 0.010 | 100% | — | — | — | — |
| | | 0.100 | 100% | — | — | — | — |
| | | 1.000 | 75% | — | — | — | — |
| | ADBAC | 0.010 | | 0% | 0% | 100% | 0% |
| | | 0.100 | | 0% | 0% | 0% | 0% |
| | | 1.000 | | 0% | 0% | 0% | 0% |
| | BZT | 0.010 | 0% | 0% | 0% | 50% | 0% |
| | | 0.100 | 0% | 0% | 0% | 0% | 0% |
| | | 1.000 | 100% | 0% | 0% | 0% | 0% |
| | BTBAC | 0.010 | 100% | — | — | — | — |
| | | 0.100 | 100% | — | — | — | — |
| | | 1.000 | 100% | — | — | — | — |
| | BDAC | 0.010 | 0% | 0% | 0% | 100% | 0% |
| | | 0.100 | 0% | 0% | 0% | 25% | 0% |
| | | 1.000 | 0% | 0% | 0% | 0% | 0% |
| Water + | TMSAC | 0.010 | 100% | 100% | 0% | | 0% |
| QAH | | 0.100 | 100% | 50% | 0% | | 0% |
| | | 1.000 | 50% | 0% | 0% | | 0% |
| | BTEAC | 0.010 | 100% | — | — | — | — |
| | | 0.100 | 100% | — | — | — | — |
| | | 1.000 | 100% | — | — | — | — |
| | ADBAC | 0.010 | | 0% | 0% | 100% | 0% |
| | | 0.100 | | 0% | 0% | 100% | 0% |
| | | 1.000 | | 0% | 0% | 0% | 0% |
| | BZT | 0.010 | 100% | 100% | 0% | | 0% |
| | | 0.100 | 25% | 100% | 0% | | 0% |
| | | 1.000 | 0% | 100% | 0% | | 0% |
| | BTBAC | 0.010 | 100% | — | — | — | — |
| | | 0.100 | 100% | — | — | — | — |
| | | 1.000 | 100% | — | — | — | — |
| | BDAC | 0.010 | 100% | 100% | 0% | 100% | 0% |
| | | 0.100 | 0% | 25% | 0% | 75% | 0% |
| | | 1.000 | 0% | 25% | 0% | 0% | 0% |

CMC + QAH refers to a quaternary ammonium halide in combination with 0.237% by weight NaHCO₃, 0.95% by weight Na₂CO₃, 1.19% by weight Na₃PO₄

### Legend

| QAH | | |
|---|---|---|
| Acronym | QAH Full Name | CAS #: |
| TMSAC | Trimethylstearylammonium chloride | 112-03-8 |
| BTEAC | Benzyltriethylammonium chloride | 56-37-1 |
| ADBAC | Alkyldimethylbenzylammonium chloride | 8001-54-5 |
| BZT | Benzethonium chloride | 121-54-0 |
| BTBAC | Benzyltri-n-butylammonium chloride | 55628-54-1 |
| BDAC | Benzyl-dimethylhexadecylammonium | 122-18-9 |
| | chloride | |

The results show that most selected QAH's are suitable substitutes for CTAB and work effectively to kill the targeted organisms within the 10-minute contact time. Specifically, TMSAC, ADBAC, BZT, and BDAC all demonstrated remarkable effectiveness against the target organisms at low concentrations of 0.01% QAH.

BTEAC and BTBAC were ineffective substitutes for CTAB for disinfection purposes, since even at relatively high concentrations of 1.0% QAH these were incapable of killing Staphylococcus aureus. The chemical structure of both BTEAC and BTBAC have a benzyl functional group and 3 alkyl groups with carbon chains longer than 2 carbon atoms. It is believed that the length of the carbon chains creates steric interference between the charged nitrogen atom of the ammonium group of the QAH and the organism cell membrane, thus limiting the accessibility of the QAH to the cell membrane of the bacteria/mold organism. In contrast, CTAB, TMSAC, ADBAC, BZT, and BDAC all have alkyl chains including one carbon atom or less which appears to be necessary for an effective combination of CMC +QAH.

### Example 2: Effect of CMC with Cetyltrimethylammonium Bromide Additive on Pseudomonas aeruginosa ATCC 15442, Staphylococcus aureus ATCC 6538 and Trichophyton mentagrophytes ATCC 9533, and Aspergillus niger ATCC 6275.

Samples of CMC-Plus (0.237% NaHCO₃, 0.95% Na₂CO₃, 1.19% Na₃PO₄, 0.01% CTAB) were tested for effectiveness against Pseudomonas aeruginosa ATCC #15442 and Staphylococcus aureus ATCC #6538 using the AOAC Germicidal Spray Method test protocol. Sample of CMC-Plus used was designated Lot # SII-16042016.

TABLE 5
Test Parameters for Evaluation of Bacteria Disinfection

| | |
|---|---|
| Test Substance Dilution: | Ready to use, Trigger Spray |
| Exposure Time: | 9.5 Minutes |
| Exposure Temperature: | 20 ± 1° C. |
| Number of Carriers | 60 Carriers |
| Tested/Lot: | |
| Soil Load Description: | No Organic Soil Load Required |
| Spray Instructions: | Spray 3-4 Times per carrier or Until Thoroughly Wet at an |
| | Approximate Distance of 6 to 8 Inches |
| Neutralizing Subculture | Letheen Broth + 0.07% Lecithin + 0.5% Tween 80 + 0.1% |
| Medium: | Sodium Thiosulphate |
| Agar Plate Medium: | Tryptic Soy Agar + 5% Sheep's Blood (BAP) |

### Experimental Design

A film of bacterial cells dried onto glass carriers was exposed to the test substance for the specified exposure time. Following exposure, the carriers were transferred to vessels containing neutralizing subculture medium. The subcultures were incubated and assayed for survivors. Appropriate culture purity, viability, neutralizing subculture medium sterility, carrier sterility, carrier population, and neutralization confirmation controls were included.

TABLE 6
Control Results-The following results from
controls confirmed study validity:

| | Results | |
|---|---|---|
| | Pseudomonas | Staphylococcus |
| | aeruginosa | aureus |
| Type of Control | (ATCC 15442) | (ATCC 6538) |
| Purity Control | Pure | Pure |
| Viability Control | Growth | Growth |

| | |
|---|---|
| Neutralizing Subculture | No growth |
| Medium Sterility Control | |
| Carrier Sterility Control | No Growth |

TABLE 7
Carrier Population Control Results

| Test Organism | Carrier Set | CFU/carrier^{a} | Log₁₀ | Average Log₁₀ |
|---|---|---|---|---|
| Pseudomonas | Pre-testing | 1.3 × 10⁵ | 5.11 | 5.05 |
| aeruginosa | Post-testing | 9.5 × 10⁴ | 4.98 | |
| (ATCC 15442) | | | | |
| Staphylococcus | Pre-testing | 3.4 × 10⁵ | 5.53 | 5.52 |
| aureus (ATCC | Post-testing | 3.17 × 10⁵ | 5.50 | |
| 6538) | | | | |

^{a}CFU = Colony forming unit

Purity Control: Used to confirm that there is no contamination in the organism strain.

Viability Control: Confirms that inoculated organisms living and viable.

Neutralizing Subculture Medium Sterility Control: Confirms that the medium used to neutralize the disinfectant is free of viable organism contamination to avoid false positive growth readings.

Carrier Sterility Control: Confirms that the carriers used in the disinfection studies are sterile and free of any viable organisms.

Pre-and post-test counting of CFU per carrier was carried out to ensure the population control is the minimum organism concentration range of log₁₀=5. Carriers were enumerated prior to test initiation to determine pre-test carrier microbial concentrations (ie. pre-test). Untreated carriers were enumerated prior to test initiation to determine post-test carrier microbial concentrations.

The average pre and post testing CFU/carrier concentration must be a minimum of log₁₀=5.

TABLE 8
Test Results

| | | | Number of Carriers | |
|---|---|---|---|---|
| | | | Inoculated with | |
| | | | Test Organism | |
| | | | and Treated | Carriers |
| | | Sample | with | Showing |
| Test Substance | Test Organism | Dilution | Disinfectant | Growth* |
| Lot SII- | Pseudomonas | Ready to Use | 60 | 0 |
| 16042016 | aeruginosa | | | |
| | (ATCC 15442) | | | |
| | Staphylococcus | | 60 | 0 |
| | aureus (ATCC | | | |
| | 6538) | | | |

*Number of carriers showing growth of test organism.

The test results indicated that 60 carriers each of Pseudomonas aeruginosa (ATCC 15442) and Staphylococcus aureus (ATCC 6538) were treated. None of the 60 carriers treated per organism showed any growth after the incubation period. These results demonstrate that CMC-Plus which comprises 0.237% by weight NaHCO₃, 0.95% by weight Na₂CO₃, 1.19% by weight Na₃PO₄, 0.01% by weight CTAB is effective for killing Pseudomonas aeruginosa (ATCC 15442) and Staphylococcus aureus (ATCC 6538).

Efficacy testing was also completed to confirm formula effectiveness against Trichophyton mentagrophytes ATCC 9533 and Aspergillus niger ATCC 6275. The protocol followed was the Fungicidal Germicidal Spray Method. Tested lot numbers were Lot # SII-05112016 (0.237% NaHCO₃, 0.95% Na₂CO₃, 1.19% Na₃PO₄, 0.025% CTAB) and Lot # SII-18092016 (0.237% NaHCO₃, 0.95% Na₂CO₃, 1.19% Na₃PO₄, 0.1% CTAB) for Trichophyton mentagrophytes ATCC 9533. Tested lot numbers were Lot # SII-05082017 (0.237% by weight NaHCO₃, 0.95% by weight Na₂CO₃, 1.19% by weight Na₃PO₄, 0.05% by weight CTAB), Lot # SII-1-28102017 (0.237% by weight NaHCO₃, 0.95% by weight Na₂CO₃, 1.19% by weight Na₃PO₄, 0.125% by weight CTAB) and Lot # SII-4-2810201 (0.237% NaHCO₃, 0.95% Na₂CO₃, 1.19% Na₃PO₄, and 0.5% CTAB) for Aspergillus niger ATCC 6275.

TABLE 9
Test Parameters for Evaluation of Fungal Disinfection

| | |
|---|---|
| Test Substance Dilution: | Ready to use, Trigger Spray |
| Exposure Time: | 9.5 Minutes |
| Exposure Temperature: | 18.8-20° C. |
| Number of Carriers | 10 Carriers |
| Tested/Lot: | |
| Soil Load Description: | No Organic Soil Load Required |
| Spray Instructions: | Spray 10 × per carrier or Until Thoroughly Wet at an |
| | Approximate Distance of 12 Inches |
| Neutralizing Subculture | Sabouraud Dextrose Broth (SBD) + 0.07% Lethicin + 0.5% |
| Medium: | Tween 80. |
| Agar Plate Medium: | Glucose Agar |

### Experimental Design

A film of fungal cells dried onto a glass surface was exposed to the test substance for the specified exposure time. Following exposure, the carriers were transferred to primary vessels containing neutralizing subculture medium. After 25-60 minutes, the carriers were transferred to secondary vessels containing neutralizing subculture medium. To pass the disinfection test, growth cannot be observed in either primary or secondary neutralization tubes. The subcultures were incubated and assayed for survivors. Appropriate culture purity, viability, neutralizing subculture medium sterility, carrier sterility, carrier population, and neutralization confirmation controls were performed.

TABLE 10
Control Results

| | Results | |
|---|---|---|
| | Trichophyton | Aspergillus |
| | mentagrophytes | Niger |
| Type of Control | ATCC 9533 | ATCC 6275 |
| Purity Control | Pure | Pure |
| Viability Control | Growth | Growth |
| Primary Neutralizing Subculture | No Growth | No Growth |
| Medium Sterility Control | | |
| Secondary Neutralizing Subculture | No Growth | No Growth |
| Medium Sterility Control | | |
| Carrier Sterility Control | No Growth | No Growth |

TABLE 11
Carrier Population Control Results

| Test | Carrier | | | Average | Tested |
|---|---|---|---|---|---|
| Organism | Set | CFU/carrier | Log₁₀ | Log₁₀ | Lot # |
| Trichophyton | Pre-testing | 7.2 × 10⁴ | 4.86 | 4.08 | Lot #'s |
| mentagrophytes | Post- | 2 × 10³ | 3.30 | | SII-18092016 |
| (ATCC 9533) | testing | | | | and SII- |
| | | | | | 05112016 |
| Aspergillus | Pre-testing | 3.2 × 10⁴ | 4.51 | 4.18 | SII- |
| Niger | Post- | 7.0 × 10³ | 3.85 | | 05082017 |
| (ATCC 6275) | testing | | | | |
| Aspergillus | Pre-testing | 2.5 × 10⁴ | 4.40 | 4.33 | SII-1- |
| Niger | Post- | 1.8 × 10⁴ | 4.26 | | 28102017 |
| (ATCC 6275) | testing | | | | |
| Aspergillus | Pre-testing | 2.5 × 10⁴ | 4.40 | 4.20 | SII-4- |
| Niger | Post- | 1.0 × 10⁴ | 4.00 | | 28102017 |
| (ATCC 6275) | testing | | | | |

Purity Control: Used to confirm that there is no contamination in the organism strain

Viability Control: Confirms that inoculated organisms living and viable.

Primary Neutralizing Subculture Medium Sterility Control: Confirms that the medium used to neutralize the disinfectant is free of viable organism contamination to avoid false positive growth readings.

Secondary Neutralizing Subculture Medium Sterility Control: Fungal test carriers are exposed to two neutralizing subculture mediums (first exposed to primary medium, then removed and placed into secondary medium for the duration of the 10 day period in which the organism has the opportunity to grow).

Carrier Sterility Control: Confirms that the carriers used in the disinfection studies are sterile and free of any viable organisms.

TABLE 12
Test Results

| | | | Number of Carriers | |
|---|---|---|---|---|
| | | | Inoculated with | |
| | | | Test Organism | Carriers |
| Test | | Sample | and Treated | Showing |
| Substance | Test Organism | Dilution | with Disinfectant | Growth* |
| CMC-Plus | Trichophyton | Ready | 10 | 1° = 0 |
| Lot SII- | mentagrophytes | to Use | | 2° = 0 |
| 18092016 | ATCC 9533 | | | |
| CMC-Plus | | | 10 | 1° = 0 |
| Lot SII- | | | | 2° = 0 |
| 05112016 | | | | |
| CMC-Plus | Aspergillus | Ready | 10 | 1° = 2 |
| Lot SII- | niger | to Use | | 2° = 0 |
| 05082017 | ATCC 6275 | | | |
| CMC-Plus | | | 10 | 1° = 1 |
| Lot SII-1- | | | | 2° = 1 |
| 28102017 | | | | |
| CMC-Plus | | | 10 | 1° = 0 |
| Lot SII-4- | | | | 2° = 0 |
| 28102017** | | | | |

*Number of carriers showing growth of the test organism.
**Disinfectant sprayed 15x per carrier rather than 10x per carrier (as per Table 9) to ensure sufficient wetting.
1° = Primary subculture
2° = Secondary subculture

The test results indicated that 10 carriers of Trichophyton mentagrophytes ATCC 9533 were treated with the disinfectant. None of the 10 carriers treated showed any growth after the incubation period. These results demonstrate that CMC-Plus compositions comprising 0.237% by weight NaHCO₃, 0.95% by weight Na₂CO₃, 1.19% by weight Na₃PO₄, 0.025% by weight CTAB (Lot SII-18092016) and .237% by weight NaHCO₃, 0.95% by weight Na₂CO₃, 1.19% by weight Na₃PO₄, 0.1% by weight CTAB (Lot SII-05112016) are effective for killing Trichophyton mentagrophytes ATCC 9533.

The results indicated that when 10 carriers inoculated with Aspergillus niger were treated with the disinfectant, only the experiment where carriers with CMC-Plus Lot # SII-4-2810201 (0.237% NaHCO₃, 0.95% Na₂CO₃, 1.19% Na₃PO₄, and 0.5% CTAB) successfully killed all fungal organisms on the treated carriers. Carriers treated with Lot # SII-05082017 (0.237% by weight NaHCO₃, 0.95% by weight Na₂CO₃, 1.19% by weight Na₃PO₄, 0.05% by weight CTAB) and Lot SII-1-28102017 (0.237% by weight NaHCO₃, 0.95% by weight Na₂CO₃, 1.19% by weight Na₃PO₄, 0.125% by weight CTAB) were unsuccessful in killing all Aspergillus niger ATCC 6275 organisms. It is noted that disinfectants featuring QAH's require higher concentrations of QAH and/or extended contact times (>10 mins) in order to disinfect organisms of the Aspergillus genus (Ohta, S., Makino, M., Nagai, K., Zenda, H. Biocontrol Science, 1999, 4(1) 41-44; Gupta, A. K., Ahad, I., Summerbell, R. C. Medical Mycology, 2002, 40, 201-208).

It is well known that it is challenging to disinfect surfaces contaminated with organisms of the Aspergillus genus, including Aspergillus niger, due to the specific qualities of the cell membrane of those organisms. The overall results show consistency in effectiveness of disinfecting surfaces contaminated with Aspergillus niger with compositions having at least 0.5% by weight QAH in combination with CMC. It is expected that higher concentrations of QAH would be required to disinfect surfaces contaminated with Aspergillus genus. The concentration level of QAH of 0.5% used in combination with CMC in order to disinfect surfaces contaminated with Aspergillus genus is significantly lower than would have been expected to disinfect such a surface with a QAH alone.

### Example 3: Reduction in CMC Concentration and Combination with Various Concentrations of CTAB and Corresponding Effectiveness Against Bacterial and Fungal Strains

A further series of experiments was conducted where the CMC concentration was decreased to 0.5 times and 0.75 times the nominal concentration of 0.237% sodium bicarbonate, 0.95% sodium carbonate, and 1.19% trisodium phosphate. Included in the CMC dilution series was cetyltrimethylammonium bromide at concentrations of 0.01%, 0.005%, and 0.001%. All percentages referred to are by weight of the composition. These solutions were tested on Staphylococcus aureus, Pseudomonas aeruginosa, and Trichophyton mentagrophytes following the AOAC Official Method 961.02 protocol used to determine disinfection potential. The results are listed in Table 13 below:

TABLE 13
Effect of CMC Concentration Reduction in Formulas
Containing CTAB and the Resulting Disinfection Rate

| | Staphylococcus | Pseudomonas | Trichophyton |
|---|---|---|---|
| | aureus | aeruginosa | mentagrophytes |
| Formulae | Growth Rate | Growth Rate | Growth Rate |
| Control-0.85% | 100% | 100% | 100% |
| NaCl | | | |
| Control-0.1% | 0 | 0 | 0 |
| CTAB in 1 × CMC | | | |
| 0.01% CTAB in | 0 | 0 | 0 |
| 0.5 × CMC | | | |
| 0.005% CTAB in | 25% | 0 | 0 |
| 0.5 × CMC | | | |
| 0.001% CTAB in | 100% | 0 | 0 |
| 0.5 × CMC | | | |
| 0.01% CTAB in | 0 | 0 | 0 |
| 0.75 × CMC | | | |
| 0.005% CTAB in | 50% | 0 | 0 |
| 0.75 × CMC | | | |
| 0.001% CTAB in | 100% | 0 | 0 |
| 0.75 × CMC | | | |

There are several concentration combinations that demonstrate effectiveness against Pseudomonas aeruginosa and Trichophyton mentagrophytes. It was noted in Table 13 above that a combination of 0.005% CTAB and 0.5×CMC was more effective at killing Staphylococcus aureus than a solution containing 0.005% CTAB and 0.75×CMC. While this may appear to be counterintuitive, it is noted that in effect the CTAB concentration has been reduced as a percentage of the overall formula. From Tables 2 and 3, it has been observed that Staphylococcus aureus kill rates increase proportionately to an increase in CTAB concentration. The results listed in Table 13 demonstrates this dose-response effect.

### Example 4: Extended Long Term Antimicrobial Activity of Compositions Comprising CMC and CTAB

Compositions comprising CMC and CTAB in concentrations of 0.01% and 0.5% by weight of the composition were tested for long term efficacy in preventing growth of Aspergillus niger and Penicillium variable after application of the composition to a surface.

The Hard Surface Mildew Fungistatic Test method was used to evaluate the mildew growth resistance of treated ceramic tiles. In this method, ceramic tiles (carriers) were treated with the antimicrobial product and the carriers were allowed to dry. Following drying, the carriers were inoculated with Aspergillus niger. The treated carriers were incubated in a high humidity environment and were visually rated for mold growth. For a product to be considered an effective mildew fungistat, the treated surfaces must demonstrate no mold growth following the incubation period of seven days.

A CMC-Plus solution (0.237% NaHCO₃, 0.95% Na₂CO₃, 1.19% Na₃PO₄, and 0.5% CTAB) was used as the antimicrobial product. After a 24-day incubation period, no growth was observed on top of the CMC-Plus treated ceramic tiles. Untreated ceramic tiles showed growth of Aspergillus niger. This confirms the antimicrobial nature of the film applied to the surface and passes the EPA criteria for Hard Surface, Mildewstat claims.

A CMC-Plus solution (0.237% NaHCO₃, 0.95% Na₂CO₃, 1.19% Na₃PO₄, and 0.01% CTAB) was also used as the antimicrobial product. After a 7-day incubation period, no growth was observed on top of the CMC-Plus treated ceramic tiles. Untreated ceramic tiles showed growth of Aspergillus niger. This confirms the antimicrobial nature of the film applied to the surface and passes the EPA criteria for Hard Surface, Mildewstat claims.

The Fabric Mildewstat Test method is analogous to the Hard Surface method whereby the carrier (in this case cotton muslin strips) were treated with the antimicrobial solution, and then inoculated with a mixture of Aspergillus niger and Penicillium variable. The treated carriers were incubated in a high humidity environment and were visually rated for mold growth. For a product to be considered an effective mildew fungistat on fabrics, the treated surfaces must demonstrate no mold growth following the incubation period of 28 days.

A CMC-Plus solution (0.237% NaHCO₃, 0.95% Na₂CO₃, 1.19% Na₃PO₄, and 0.5% CTAB) was used as the antimicrobial product. After 28 days, the cotton carriers were inspected and showed no signs of mold growth, thereby passing the EPA criteria for a Fabric Mildewstat claim.

In addition, a CMC-Plus solution (0.237% NaHCO₃, 0.95% Na₂CO₃, 1.19% Na₃PO₄, and 0.01% CTAB) was used as the antimicrobial product. After 28 days, the cotton carriers were inspected and showed no signs of mold growth, thereby passing the EPA criteria for a Fabric Mildewstat claim.

The scope of the claims should not be limited by the preferred embodiments set forth in the examples, but should be given the broadest interpretation consistent with the description as a whole.

1. An aqueous disinfectant composition, comprising from about 0.25% to about 3.0% by weight of trisodium phosphate, from about 0.25% to about 3.0% by weight of sodium carbonate, from about 0.1% to about 3.0% by weight of sodium bicarbonate, from about 0.01% by weight to about 0.1% by weight of a quaternary ammonium halide.

2. The aqueous disinfectant composition according to claim 1 wherein the composition comprises from about 0.05% to about 0.1% by weight of the quaternary ammonium halide.

3. The aqueous disinfectant composition according to claim 1 wherein the quaternary ammonium halide is cetyltrimethylammonium bromide or alkyldimethylbenzylammonium chloride.

4. The aqueous disinfectant composition according to claim 1 wherein the quaternary ammonium halide is cetyltrimethylammonium bromide.

5. The aqueous disinfectant composition according to claim 4 wherein the composition comprises from about 0.05% to about 0.1% by weight of cetyltrimethylammonium bromide.

6. The aqueous disinfectant composition according to claim 1 wherein the quaternary ammonium halide is selected from the group consisting of cetyltrimethylammonium bromide, benzalkonium chloride, trimethylstearylammonium chloride, alkyldimethylbenzylammonium chloride, benzethonium chloride and benzyl-dimethylhexadecylammonium chloride.

7. The aqueous disinfectant composition according to claim 6 wherein the composition comprises from about 0.05% to about 0.1% by weight of the quaternary ammonium halide.

8. The aqueous disinfectant composition according to claim 1 wherein the composition comprises 0.237% by weight of sodium bicarbonate, 0.948% by weight of sodium carbonate and 1.185% by weight of trisodium phosphate.

9. The aqueous disinfectant composition according to claim 2 wherein the composition comprises 0.237% by weight of sodium bicarbonate, 0.948% by weight of sodium carbonate and 1.185% by weight of trisodium phosphate.

10. An aqueous disinfectant composition, comprising about 0.237% by weight of sodium bicarbonate, about 0.948% by weight of sodium carbonate, about 1.185% by weight of trisodium phosphate and about 0.01% to about 0.1% by weight of cetyltrimethylammonium bromide.

11. An aqueous disinfectant composition, comprising from about 0.25% to about 3.0% by weight of trisodium phosphate, from about 0.25% to about 3.0% by weight of sodium carbonate, from about 0.1% to about 3.0% by weight of sodium bicarbonate and a quaternary ammonium halide.

12. Use of a composition, comprising about 0.237% by weight of sodium bicarbonate, about 0.948% by weight of sodium carbonate, about 1.185% by weight of trisodium phosphate and from about 0.010% to about 0.1% by weight of a quaternary ammonium halide in the preparation of an aqueous disinfectant formulation.

13. The use according to claim 12 wherein the quaternary ammonium halide is selected from the group consisting of cetyltrimethylammonium bromide, trimethylstearylammonium chloride, alkyldimethylbenzylammonium chloride, benzethonium chloride and benzyl-dimethylhexadecylammonium chloride.

14. The use according to claim 12 wherein the quaternary ammonium halide is cetyltrimethylammonium bromide.

15. The use according to claim 14 wherein the composition comprises from about 0.05% to about 0.1% by weight of cetyltrimethylammonium bromide.

16. Use of a composition, comprising from about 0.25% to about 3.0% trisodium phosphate, from about 0.25% to about 3.0% sodium carbonate, 0.1% to about 3.0% sodium bicarbonate and about 0.5% by weight of a quaternary ammonium halide in the preparation of an aqueous disinfectant formulation for disinfecting a surface contaminated with organisms of Aspergillus genus.

17. The use according to claim 16 wherein the quaternary ammonium halide is selected from the group consisting of cetyltrimethylammonium bromide, benzalkonium chloride, trimethylstearylammonium chloride, alkyldimethylbenzylammonium chloride, benzethonium chloride and benzyl-dimethylhexadecylammonium chloride.

18. The use according to claim 16 wherein the quaternary ammonium halide is cetyltrimethylammonium bromide.

19. The use according to claim 18 wherein the composition comprises about 0.237% by weight of sodium bicarbonate, about 0.948% by weight of sodium carbonate, about 1.185% by weight of trisodium phosphate.

20. The use according to claim 19 wherein the composition comprises from about 0.5% to about 1% by weight of cetyltrimethylammonium bromide.

21. A composition comprising about 0.237% by weight of sodium bicarbonate, about 0.948% by weight of sodium carbonate, about 1.185% by weight of trisodium phosphate and about 0.5% by weight of a quaternary ammonium halide for disinfecting a surface contaminated with organisms of Aspergillus genus.
